(19) United States
(12) Patent Application Publication
(10) Pub. No.: US 2026/0056207 A1
(43) Pub. Date: Feb. 26, 2026

(54) METHOD OF DETERMINING NEUTRALIZING ANTIBODIES USING MODIFIED HEMAGGLUTINATION ASSAYS

(71) Applicant: VST LLC dba MEDGENE, Brookings, SD (US)

(72) Inventors: Sherry R. Glover, Brookings, SD (US); Alan John Young, Brookings, SD (US)

(73) Assignee: VST LLC dba Medgene, Brookings, SD (US)

(21) Appl. No.: 19/212,439

(22) Filed: May 19, 2025

Related U.S. Application Data
(60) Provisional application No. 63/648,832, filed on May 17, 2024.

(51) Int. Cl. G01N 33/68; C07K 14/005; C12N 15/86; G01N 33/569

(52) U.S. Cl. CPC G01N 33/6854; C07K 14/005; C12N 15/86; G01N 33/56983; C12N 2710/14043; C12N 2760/16122; G01N 2333/11; G01N 2469/20

(57) ABSTRACT

The present invention describes hemagglutination inhibition (HAI) assay methods to determine candidate subunit protein vaccines in the absence of using source viruses.

### CROSS REFERENCE TO RELATED APPLICATION

This application claims benefit under 35 U.S.C. § 119(e) to U.S. Provisional Application No. 63/648,832, filed May 17, 2024, which is incorporated by reference herein in its entirety.

### BACKGROUND OF THE INVENTION

### Field of the Invention

The present invention relates generally to immune-diagnostics, and more specifically to the use of hemagglutination and hemagglutination inhibition assays to determine serum antibody titer with subunit protein antigens to identify neutralizing antibodies in the absence of native or inactivated virus.

### Background Information

Certain viruses can agglutinate the red blood cells inside specific animals, which is defined as viral hemagglutination. A hemagglutination assay (HA) is designed to speculate a virus in the tested materials and test whether this reaction is specific. However, the ability of a virus to agglutinate red blood cells can be inhibited by corresponding particular antibodies, which lead to the development of hemagglutination inhibition assay (HAI).

Through the HA-HAI test, a known serum can be used to identify unknown viruses, and known viruses can also be applied to detect the contents of corresponding antibodies and titrate antibodies in the unknown serum. Hemagglutination assay is used in combination with interactions to create visible networks that indicate relative viral and antiviral antibody concentrations.

Hemagglutination assay is a mature method widely used in the influenza vaccine, diagnostic, and surveillance communities to measure virus and antibody titers and monitor influenza subtypes. Hemagglutination occurs when the hemagglutinin protein on the surface of the influenza virus binds to sialic acid on red blood cells (RBCs). Similarly, with a sufficient number of viruses and red blood cells, the virus particles act as bridging agents to create a network of linked RBCs, thereby causing hemagglutination.

Hemagglutination assay is the most commonly used method for influenza virus, adenovirus, rabies virus, and many other micro-organisms detection. It is the basis of a wide range of veterinary and life science diagnostic assay.

Because of the difficulty and costs associated with the identification of the source virus, viral isolation, means of growing said virus, including handling of high biosecurity (e.g., highly pathogenic avian influenza (HPAI)) viruses, what is needed is an assay that overcomes the need to use source virus.

### SUMMARY OF THE INVENTION

The present invention relates to method of identifying candidate subunit proteins derived from viral antigens that will produce neutralizing antibodies when used in a vaccine.

In embodiments, a hemagglutination inhibition assay (HIA) is disclosed including mixing sera containing antibody, a subunit protein derived from an avian influenza viral antigen, and red blood cells (RBCs) in the absence of live or attenuated avian influenza virus (IAV); allowing the mixture to stand for a sufficient time to allow for interaction between the three components; and determining whether the RBCs aggregate.

In one aspect, if aggregation of the RBCs is inhibited, then the subunit protein will produce neutralizing antibodies when used in a vaccine composition. In another aspect, the subunit protein is an avian influenza surface antigen. In a related aspect, the subunit protein is hemagglutinin and/or neuraminidase.

In another aspect, the IAV strain is a highly pathogenic avian influenza (HPAI) type A or type B strain. In a related aspect, the IAV includes type B influenza, subtype H₃N₂, type A influenza, subtype H₁N₁, type A influenza, subtype H₅N₁, type A influenza, subtype H₅N₂, type A influenza, subtype H₉N₂, type A influenza, subtype H₆N₁, and type A influenza, subtype H₇N₇.

In one aspect, the subunit protein is obtained from A/Common tern/ME/22-020831-002 original/2022 (H₅N₁) (GenBank No.: PQ709561.1). In a related aspect, the subunit protein is screened against antibody generated from IAV strains including A/swine/Missouri/A01410818/2013 (H₃N₁) (GenBank No.: KJ941368.1), A/swine/Nebraska/A02245325/2019 (H₃N₂) (GenBank No.: MN932087.1), A/swine/Iowa/A01432233/2012 (H₁N₁) (GenBank No.: KC436080.1), A/swine/Iowa/A02478455/2019 (H₃N₂) (GenBank No.: MK967587.1), A/swine/Missouri/A01410818/2013 (H₃N₁) (GenBank No.: KJ941268.1), A/swine/Ohio/A02431145/2019 (H₁N₂) (GenBank No.: MK714853.1), A/swine/North Carolina/A02431520/2019 (H₁N₂) (GenBank No.: MK799887.1), A/swine/North Carolina/A02479173/2020 (H₁N₁) (GenBank No.: MT367647.1), A/West Virginia/04/2020 (H₁) (GenBank No.: MT330463.1), A/swine/Iowa/A02479163/2020 (H₁N₂) (GenBank No.: MT476698.1), and A/swine/Iowa/A01566613/2014 (H₃N₂) (GenBank No.: KP247601.1).

In one aspect, the antibody is collected from the sera of animals including swine, bovine, sheep, rabbit, deer, and equine subjects, where said animal is infected by an IAV or naïve to such infection.

Tin another aspect, a neutralizing antibody is positively identified if it has an HA assay titer of at least 320 HAU/50 μl.

In embodiments, a hemagglutination inhibition assay (HIA) for identifying neutralizing antibodies against avian influenza virus (IAV) is disclosed including mixing sera containing antibody, a subunit protein derived from an avian influenza viral antigen, and red blood cells (RBCs) in a suitable container in the absence of live or attenuated IAV, where the subunit protein is generated by recombinant means; allowing the mixture to stand for a sufficient time to allow for interaction between the three components; and determining whether the RBCs aggregate.

In one aspect, the recombinant means is a recombinant baculovirus protein expression system including infecting Spodoptera frugiperda insect cells (Sf9) with baculovirus containing heterologous nucleic acids encoding said subunit protein, where a final subunit protein sequence is optimized for expression in Sf9. In a related aspect, the resulting viral fluids generated from infected Sf9 cells are inactivated with Beta-propiolactone (BPL). In a further related aspect, the sera is combined with receptor destroying enzyme (RDE) or kaolin.

In one aspect, the RBCs are from turkey.

In another aspect, the IAV strain is a highly pathogenic avian influenza (HPAI) type A or type B strain. In a related aspect, the IAV includes type B influenza, subtype H₃N₂, type A influenza, subtype H₁N₁, type A influenza, subtype H₅N₁, type A influenza, subtype H₅N₂, type A influenza, subtype H₉N₂, type A influenza, subtype H₆N₁, and type A influenza, subtype H₇N₇.

In one aspect, the subunit protein is obtained from A/Common tern/ME/22-020831-002 original/2022 (H₅N₁) (GenBank No.: PQ709561.1). In a related aspect, the subunit protein is screened against antibody generated from IAV strains including A/swine/Missouri/A01410818/2013 (H₃N₁) (GenBank No.: KJ941368.1), A/swine/Nebraska/A02245325/2019 (H₃N₂) (GenBank No.: MN932087.1), A/swine/Iowa/A01432233/2012 (H₁N₁) (GenBank No.: KC436080.1), A/swine/Iowa/A02478455/2019 (H₃N₂) (GenBank No.: MK967587.1), A/swine/Missouri/A01410818/2013 (H₃N₁) (GenBank No.: KJ941268.1), A/swine/Ohio/A02431145/2019 (H₁N₂) (GenBank No.: MK714853.1), A/swine/North Carolina/A02431520/2019 (H₁N₂) (GenBank No.: MK799887.1), A/swine/North Carolina/A02479173/2020 (H₁N₁) (GenBank No.: MT367647.1), A/West Virginia/04/2020 (H₁) (GenBank No.: MT330463.1), A/swine/Iowa/A02479163/2020 (H₁N₂) (GenBank No.: MT476698.1), and A/swine/Iowa/A01566613/2014 (H₃N₂) (GenBank No.: KP247601.1), and where the antibody is collected from the sera of animals including swine, bovine, sheep, rabbit, deer, and equine subjects, and where the animal is infected by an IAV or naïve to such infection.

In one aspect, a neutralizing antibody is positively identified if it has an HA assay titer of at least 320 HAU/50 μl.

### DETAILED DESCRIPTION OF THE INVENTION

Before the present composition, methods, and methodologies are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “a nucleic acid” includes one or more nucleic acids, and/or compositions of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Any methods and materials similar or equivalent to those described herein can be used in the practice or testing of the invention, as it will be understood that modifications and variations are encompassed within the spirit and scope of the instant disclosure.

As used herein, “about,” “approximately,” “substantially” and “significantly” will be understood by a person of ordinary skill in the art and will vary in some extent depending on the context in which they are used. If there are uses of the term which are not clear to persons of ordinary skill in the art given the context in which it is used, “about” and “approximately” will mean plus or minus <10% of particular term and “substantially” and “significantly” will mean plus or minus >10% of the particular term. In embodiments, composition may “contain,” “comprise,” or “consist essentially of” a particular component or group of components, where the skilled artisan would understand the latter to mean the scope of the claim is limited to the specified materials or steps “and those that do not materially affect the basic and novel characteristic(s)” of the claimed invention.

“Antigens” are molecular structures on the surface of viruses that are recognized by the immune system and are capable of triggering one kind of immune response known as antibody production. Two proteins (hemagglutinin and neuraminidase) on the surface of influenza viruses contain the major antigens targeted by antibodies.

When a subject (human or animal) is exposed to an influenza virus (either through infection or vaccination) their immune system makes specific antibodies against the antigens on that particular influenza virus. The term “antigenic properties” is used to describe the immune response triggered by the antigens on a particular virus. “Antigenic characterization” refers to the analysis of a virus' antigenic properties to help assess how related it is to another virus.

Avian influenza virus titer is commonly assayed using live virus, depending on the purpose of the assay. Several methods exist to determine the titer (i.e., the concentration of infectious virus), and the most appropriate one depends on the context—vaccine development, research or diagnosis.

The main assay types used to determine avian influenza virus titer are as follows:
Hemaggultination (HA) Assay: Influenza viruses have hemagglutinin proteins that bind to sialic acid receptors on red blood cells (RBCs), causing agglutination.
   Rapid, inexpensive estimate of total viral particles (not necessarily infectious)
   Live virus-containing allantoic acid fluid or culture supernatant
   Does not distinguish between infectious and non-infectious virus particles
Hemaggultination Inhibitions (HI) Assay: Measures the ability of antibodies to prevent HA from binding to RBCs
   Common for vaccine potency or serological testing, not direct virus titer per se
   Virus and sera from immunized animals
Egg Infectious Dose 50 (EID₅₀): Serial dilutions of virus are inoculated into embryonated chicken eggs. After incubation, the presence of virus is confirmed via HA assay.
   The dilution at which 50% of the eggs are infected
   Considered the gold standard for live avian virus quantification
   Requires biosafety-level handling (typically BSL-3 for H5 viruses)
Tissue Culture Infectious Dose 50 (TCID₅₀): Virus form plaques (zones of infected/destroyed cells) on a monolayer of susceptible cells.
   Accurate quantification of infectious virus (plaque-forming units/ml)
   Live virus; must form viable plaques
qRT-PCR: Often uses in parallel but does not measure infectivity—only quantifies viral RNA, so it is useful for qualifying load but not titer in the classical infectious sense.

TABLE 1
Summary of Assay Types

| Assay | | Live | | |
|---|---|---|---|---|
| Type | Measures | Virus | Infectivity | Use |
| HA Assay | HA units | Yes | No | Screening, quantifying |
| | | | | total particles |
| HI Assay | Antibody titer | Yes | Indirect | Imunogenicity/vaccine |
| | | | | studies |
| EID₅₀ | Infectious virus | Yes | Yes | Vaccine production, |
| | | | | virus quantification |
| TCID₅₀ | Infectious virus | Yes | Yes | Cell culture virus titer |
| Plaque assay | Infectious unit | Yes | Yes | High resolution |
| | | | | tittering |
| qRT-PCR | Viral RNA | No | No | Diagnostic, research |
| | copies | | | (not titer) |

As see in Table 1, except for qRT-PCR, all assay types for avian influenza require the use of live virus to determine titer. Specifically, HI, which quantifies virus specific antibodies, and which screens for virus neutralizing antibodies against HA proteins in serum, uses live or inactivated virus.

Typically, a test called the hemagglutination inhibition assay (HI test) is used to antigenically characterize influenza viruses. Hemagglutinin proteins on the surface of influenza viruses can bind to red blood cells and “glue” them together, forming a lattice structure (i.e., “hemagglutination”). In embodiments, the HI assay as disclosed herein, is independent from the use of live/inactivated virus and uses subunit HA protein as a substitute.

A plate type that is used that can clearly distinguish between positive and negative agglutination, which may vary depending on the type of red blood cell. For example, when using untreated red blood cells for analysis, a U-shaped plate may be used instead of a V-shaped plate.

In one aspect, trypsinized glutaraldehyde-fixed rabbit red blood cells may be used because trypsin treatment makes the cells more sensitive to agglutination, and glutaraldehyde fixation allows the cells to be stored for long periods (>1 month).

In another aspect, erythrocytes from different species (chicken, guinea pig, goose, horse, cow, sheep, rabbit, and human types A, B, AB, and O) may be used as reference standards to standardize the experiment.

Because the aggregation capacity of red blood cells may decrease over time, fresh red blood cells may be used.

HA analysis is simple and uses relatively inexpensive and commonly used instruments and supplies, and results can be provided within hours. Typical HA tests involve two main components: virus and red blood cells that are mixed together in the wells of a microtiter plate.

For an influenza HA test, red blood cells (RBCs) are taken from animals (e.g., turkeys), which RBCs are used to bind to influenza viruses. Normally, RBCs in a solution will sink to the bottom of the microtiter plate well and form a red dot at the bottom. However, when an influenza virus is added to the RBC solution, the virus' hemagglutinin (HA) surface proteins will bind to multiple RBCs. When influenza viruses bind to the RBCs, the RBCs form a lattice structure. This keeps the RBCs suspended in solution instead of sinking to the bottom and forming the red dot (hemagglutination, may be determined by Optical Density at, e.g., 540 nm).

The HI analysis works by measuring how well antibodies bind to the HA proteins and prevent them from “gluing” red blood cells together (i.e., hemagglutination inhibition).

HI test may be used to assess the antigenic similarity between different influenza viruses. This test helps to select candidate vaccine viruses (CVVs), which can then be included in seasonal flu vaccines. HI test results can determine whether antibodies developed after vaccination (or infection) with one virus can recognize and bind to other viruses, which means these other viruses are similar to the vaccine virus. Scientists also use the HI test to compare the antigenic properties (i.e., the virus' ability to be recognized by antibodies) of currently circulating influenza viruses with those of influenza viruses that have circulated in the past.

Typical HI tests involve three main components: antibodies, virus, and red blood cells that are mixed together in the wells of a microtiter plate.

The antibodies used in the HI test are obtained by infecting an animal that is immunologically naïve (i.e., it has not been exposed to any virus or vaccine previously in its lifetime). The animal's immune system creates antibodies in response to the antigens on the surface of the specific virus that was used to infect that animal. To study these antibodies, a sample of blood is drawn from the animal, from which serum is obtained. The HI test measures how well these antibodies recognize and bind to other influenza viruses (such as, influenza viruses isolated from flu patients). If the subject antibodies that resulted from exposure to the vaccine virus recognize and bind well to the influenza virus from a sick subject, this indicates that the vaccine virus is antigenically similar to the virus obtained from the sick patient. This finding has implications for how well the vaccine might work.

For example, influenza viruses used in the HI test may be obtained from sick subjects. Centers collect specimens from subjects all over the world to track which influenza viruses are infecting subject populations and to monitor how these viruses are changing.

For an influenza HI test, red blood cells (RBCs) are taken from animals (e.g., turkeys), which RBCs are used to bind to influenza viruses. Normally, RBCs in a solution will sink to the bottom of the microtiter plate well and form a red dot at the bottom. However, when an influenza virus is added to the RBC solution, the virus' hemagglutinin (HA) surface proteins will bind to multiple RBCs. When influenza viruses bind to the RBCs, the RBCs form a lattice structure (hemagglutination).

When antibodies are pre-mixed with influenza virus followed by RBCs, the antibodies will bind to influenza virus antigens that they recognize, covering the virus so that its HA surface proteins can no longer bind to RBCs. The reaction between the antibody and the virus inhibits hemagglutination from occurring, which results in hemagglutination inhibition.

Hemagglutination occurs when antibodies do not recognize and bind to the influenza viruses in the solution, and as a result, the influenza viruses bind to the red blood cells, forming the lattice structure. When the antibodies do recognize and bind to the influenza virus in the solution, this shows that the vaccine virus is similar to the influenza virus obtained from the sick subject. When this happens, the influenza virus being tested is said to be “antigenically similar” to the influenza virus that created the antibodies in the infected, immunologically naïve animal.

When a circulating influenza virus is antigenically different from a vaccine, the antibodies developed in response to the vaccine virus may not recognize and bind this virus. In the HI test, this will cause hemagglutination to occur. Circulating influenza viruses tested via the HI test may be obtained from respiratory samples collected from sick subjects.

In embodiments, HA tests of the invention as disclosed involve two main components: subunit protein (i.e., one or more viral antigens) obtained from baculovirus expression (sHA) and red blood cells that are mixed together in the wells of a microtiter plate, using virus-red blood cell combinations as controls.

For an influenza HA test, red blood cells (RBCs) are taken from animals (e.g., turkeys), which RBCs are used to bind to the sHA. Again, in the absence of virus (control) or sHA, RBCs in a solution will sink to the bottom of the microtiter plate well and form a red dot at the bottom. However, when an influenza virus or sHA is added to the RBC solution, the virus' hemagglutinin (HA) surface proteins or sHA will bind to multiple RBCs. When influenza viruses or sHA bind to the RBCs, the RBCs form a lattice structure (hemagglutination).

In embodiments, HI tests of the invention as disclosed involve three main components: antibodies, sHA, and red blood cells that are mixed together in the wells of a microtiter plate. Again, virus may be used in place of sHA as a control.

For an influenza HI test, red blood cells (RBCs) are taken from animals (e.g., turkeys), which RBCs are used to bind to sHA or influenza viruses (control). Again, RBCs in a solution will sink to the bottom of the microtiter plate well and form a red dot at the bottom. However, when sHA or an influenza virus (control) is added to the RBC solution, the sHA or virus' HA surface proteins will bind to multiple RBCs. When the sHA or influenza viruses bind to the RBCs, the RBCs form a lattice structure (hemagglutination).

When antibodies are pre-mixed with the sHA or influenza virus (control) followed by RBCs, the antibodies will bind to the sHA or influenza virus antigens that they recognize, covering the sHA or virus so that neither the sHA nor viral HA surface proteins can bind to RBCs. The reaction between the antibody and the sHA or virus inhibits hemagglutination from occurring, suggesting that the sHA is a good vaccine candidate (i.e., will produce neutralizing antibodies when used in a vaccines). If hemagglutination is observed, that sHA is not such a candidate.

High affinity antibody directed against a particular HA antigen can typically inhibit hemagglutination despite being diluted significantly (e.g., after a 1 to 2056 dilution). Antibodies directed at a similar related HA antigen can still bind, but not as efficiently, such that fewer dilutions can be done before hemagglutination is abrogated. A four-fold difference in titer in the HI assay is typically taken as a reasonable approximation of antigenic drift sufficient to interrupt immunity. If the titer is less than 2, the antibodies cannot bind and/or cannot neutralize their target. Antigenic maps of the relationships between viruses can be constructed in this manner.

The present invention provides methods for the identification of antigens that may be used in the prevention of viral infections, including viral influenza infections. Antigens of the invention include antigenic components of influenza viruses useful in vaccines. In embodiments, compositions of the invention include an immunogenic composition comprising viral component antigen in the form of a subunit protein. In one aspect, the antigenic component includes hemagglutinin proteins, or antigenic portions thereof.

In embodiments, the target population of influenza virus strains from which the component antigen is derived includes influenza virus isolates of an individual type. In embodiments, the target population of influenza virus strains includes influenza virus isolates of an individual subtype. In yet another embodiment, the target population of influenza virus strains includes influenza virus isolates of a geographic region. Exemplary influenza virus strains include, but are not limited to, type B influenza, subtype H₃N₂, type A influenza, subtype H₁N₁, type A influenza, subtype H₅N₁, type A influenza, subtype H₅N₂, type A influenza, subtype H₉N₂, type A influenza, subtype H₆N₁, and type A influenza, subtype H₇N₇.

The term “antigenic component(s)” in reference to influenza viruses includes, for example, viral transmembrane and surface proteins and glycoproteins, such as HA and neuraminidase (NA). The term also encompasses antigenic portions of viral proteins and glycoproteins (including conserved T cell epitopes).

The immunogenic compositions disclosed herein are designed to elicit both T cell and B cell responses against antigens representative of the antigenic diversity of a target population of influenza virus strains. In specific embodiments, the antigenic component-encoding sequences are contained in baculovirus constructs, which when expressed in Sf9 cells can be used as a vaccine, as well as a source of recombinant protein for subsequent protein boosts.

Methods for inducing an immune response in a subject against influenza viruses are further provided. These methods include administering to a subject a therapeutically effective amount of the immunogenic compositions disclosed herein.

Influenza viruses belong to the Orthomyxoviridae family and are enveloped, negative-stranded RNA viruses. They are classified as influenza types A, B, and C, of which influenza A is the most pathogenic and is believed to be the only type able to undergo reassortment within animal strains. Influenza types A, B, and C can be distinguished by differences in their nucleoprotein and matrix proteins. Influenza A subtypes are defined by variation in their HA and NA genes and usually distinguished by antibodies that bind to the corresponding proteins.

The influenza A viral genome consists of eleven genes distributed in eight RNA segments. The genes encode eleven proteins, including the envelope glycoproteins hemagglutinin and neuraminidase. Influenza A virus classification is based on the hemagglutinin (H₁-H₁₆) and neuraminidase (N₁-N₉) genes. World Health Organization nomenclature defines each virus strain by its animal host of origin (specified unless human), geographical origin, strain number, year of isolation, and antigenic description of HA and NA. See Julkunen et al., Cytokine and Growth Factor Reviews, 12:171-80, 2001 for further details regarding the influenza A virus and its molecular pathogenesis. The organization of the influenza B viral genome is similar to that of influenza A, while the influenza C viral genome contains seven RNA segments and lacks NA.

Genetic variation in influenza A occurs by two primary mechanisms. Genetic drift, which occurs via point mutations (which often occur at antigenically significant positions due to selective pressure from host immune responses), and genetic shift (also referred to as reassortment), involving substitution of a whole viral genome segment of one subtype by that of another. Many different types of animal species, including humans, swine, birds, horses, aquatic mammals, and others, can become infected with influenza A viruses. Some influenza A viruses are restricted to a particular species and will not normally infect a different species. However, some influenza A viruses may infect several different animal species, principally birds, swine, and humans. This ability is considered to be responsible for major antigenic shifts in influenza A virus. When two (or more) different viruses reproduce in the same host cell simultaneously, the genes of the two strains may “mix,” resulting in a new virus with a unique combination of RNA segments. This process is called genetic reassortment.

In embodiments, the present invention pertains to methods of identifying immunogenic compositions useful as a vaccine. As noted, the compositions include antigenic components that are selected to elicit a cross-reactive response, including the production of antibodies and/or a T cell response, against viral proteins from influenza virus strains across a target population of viruses, thus providing immunity in a subject to a number of influenza virus strains within the target population of viruses. In one aspect, a subunit protein of H₅N₁ virus origin (A/Common tern/ME/22-020831-002 original/2022 (H₅N₁) (GenBank No.: PQ709561.1)) was tested as an antigen in the HI assay for screening neutralizing antibodies to specific strains of different clades, including but not limited to, A/swine/Missouri/A01410818/2013 (H₃N₁) (GenBank No.: KJ941368.1), A/swine/Nebraska/A02245325/2019 (H₃N₂) (GenBank No.: MN932087.1), A/swine/Iowa/A01432233/2012 (H₁N₁) (GenBank No.: KC436080.1), A/swine/Iowa/A02478455/2019 (H₃N₂) (GenBank No.: MK967587.1), A/swine/Missouri/A01410818/2013 (H₃N₁) (GenBank No.: KJ941268.1), A/swine/Ohio/A02431145/2019 (H₁N₂) (GenBank No.: MK714853.1), A/swine/North Carolina/A02431520/2019 (H₁N₂) (GenBank No.: MK799887.1), A/swine/North Carolina/A02479173/2020 (H₁N₁) (GenBank No.: MT367647.1), A/West Virginia/04/2020 (H₁) (GenBank No.: MT330463.1), A/swine/Iowa/A02479163/2020 (H₁N₂) (GenBank No.: MT476698.1), and A/swine/Iowa/A01566613/2014 (H₃N₂) (GenBank No.: KP247601.1).

In a related aspect, sera from different animals is also used, including sera from, but not limited to, bovine, swine, sheep, deer, horse, rabbit, as well as fetal bovine serum. In another related aspect, the HI titers using subunit protein as an antigen demonstrates that the subunit protein antigen behaves in a manner similar to inactivated whole virus in a standard HI assay.

In embodiments, influenza subunit proteins as disclosed are based on in vitro expression of certain influenza genes that encode main structural proteins, without assembly of any nucleic acid segments which are necessary for replication, making subunit proteins safer than whole inactivated virus. In one aspect, subunit proteins may be prepared from only hemagglutinin (HA) protein without neuraminidase. In a further related aspect, subunit proteins may be used as an antigen for the HI assay without steric inhibition caused by neuraminidase which can alter titration of serum antibodies. In addition, subunit proteins without neuraminidase protein may be used for production of sera for influenza typing without interference by steric inhibition, which can occur when using sera prepared from whole virus antigen that causes false positive results. In another aspect, subunit proteins may be used as a universal antigen for the HI assay in a manner apt for quantitation of serum antibodies of different influenza subtypes. This is not always possible with whole virus antigen due to safety concerns, especially for HPAI strains. Cost is also a significant issue in diagnosis and control measures, especially in the veterinary field. In a related aspect, the cost of preparation of subunit proteins in insect cell tissue culture by using a baculovirus expression system is lower than the cost of the cultivation of whole virus in SPF eggs and the inactivation process, suggesting that recombinant subunit protein can be a cost-effective antigen for HI assays.

Virus-like particles (VLPs) are distinct from subunit protein vaccines as the former present repetitive antigenic epitopes on their surface in a confirmation that would be expected to be readily detected by the immune system. Subunit vaccines, on the other hand, are expected to have poor immunogenicity due to potential misfolding of targeted antigen or insufficient presentation to the immunological system, thus, their efficiency would be expected to be equally poor as an antigen for HI assays. As disclosed herein, the HI assay is effective with subunit protein only, in the absence of VLPs, including the absence of M1.

The term “subject,” as used herein, refers to an individual susceptible to infection with a virus, for example, an influenza virus. The term includes birds and mammals, for example, domesticated birds and mammals (such as poultry, cattle and swine), wild animals (e.g., migratory birds and deer), non-human primates, and humans.

By “immunogenic composition” is intended a composition useful for stimulating or eliciting a specific immune response (or immunogenic response) in a subject, such as a cell-mediated immune response, a humoral immune response, or both (which can originate from naïve or memory cells). An immunogenic composition can include, for example, an antigenic component (e.g., an HA protein). In embodiments, the immunogenic response is protective or provides protective immunity, in that it enables the subject to better resist infection with or disease progression from the influenza virus against which the composition is directed. One specific example of a type of immunogenic composition is a vaccine. In embodiments, a therapeutically effective amount of an immunogenic composition is administered to a subject.

A “therapeutically effective amount” of an immunogenic composition is an amount which, when administered to a subject, is sufficient to achieve a desired effect in a subject being treated with that composition. For example, this may be the amount of an immunogenic composition useful in increasing resistance to, preventing, ameliorating, and/or treating infection and disease caused by an influenza virus in a subject. Ideally, a therapeutically effective amount of an immunogenic composition is an amount sufficient to increase resistance to, prevent, ameliorate, and/or treat infection and disease caused by an influenza virus in a subject without causing a substantial cytotoxic effect in the subject. The effective amount of an immunogenic composition useful for increasing resistance to, preventing, ameliorating, and/or treating infection and disease caused by an influenza virus in a subject will depend on the subject being treated, the severity of the affliction, and the manner of administration of the immunogenic composition. Responses to a therapeutically effective amount of an immunogenic composition can include, for instance, a cell-mediated immune response, a humoral immune response, or both (which can originate from naïve or memory cells).

In embodiments, subunit proteins are made via a recombinant baculovirus protein expression system. In one aspect, the Recombinant Baculovirus protein expression system is based on a nucleic acid sequence for targeted influenza viral proteins. The final sequence is optimized for expression in Spodoptera frugiperda insect cells (Sf9) to ensure that appropriate restriction endonuclease sites are present at the termination of the sequence.

A pBacPAK8 cloning vector (Clontech Laboratories, Inc., Mountain View, CA) may be used to prepare a plasmid vector containing the target sequence. A plasmid vector contains flanking sequences homologous to the linear BestBac 2.0 Baculovirus vector (Expression Systems, Davis, CA), such that when the plasmid containing the viral HA insert was co-transfected into Sf9 cells with the linear BestBac 2.0 virus Baculovirus backbone (Expression Systems, Davis, CA), homologous recombination exchanges the H3 insert for the polyhedrin gene of the Baculovirus. The resulting Baculovirus containing the HA viral sequence expressed under control of the polyhedrin promoter is then harvested. Cells and virus are grown in culture media obtained from Expression Systems (Media ES 99-300) formulated without animal origin ingredients. Gentamicin solution is added to a final concentration of 10 μg/ml from purchased stock solution (Gibco Cat #15710). At final harvest, infected cultures are centrifuged to remove the cells and the supernatant collected. The supernatant was processed through 0.2-micron sterile disposable filter. The premaster culture is titered to determine final concentration.

Sf9 cells are scaled up to production quantities utilizing glass or sterile disposable plastic vessel volumes. Upon reaching the final cell culture volume required for production, virus infection occurs in the same vessel as the final passage of cells is prepared. Culture mixing is achieved through shaking/rocking of the container or utilizing low shear type impeller design. Mixing speed and intensity is adjusted to maintain cells in suspension without creating excess shear or foaming which will cause cell disruption.

Viral fluids are inactivated with Beta-propiolactone (BPL) at a final concentration of 0.2-0.3%. Prior to inactivation, the pH of the disrupted fluids are adjusted to 7.5-8.0 using 2-ION NaOH as base or 10-38% HCl or 10% Nitric acid as acid. The disrupted fluids are allowed to warm to room temperature for 1-18 hours prior to the addition of BPL. BPL is added at the concentration specified above, with mixing. After the addition of BPL, the viral fluids are transferred to an inactivation container utilizing a “bottom to bottom” transfer process to ensure that all fluids have come into contact with BPL. The disrupted fluids are incubated at 17-27° C. for 18-48 hours with agitation. After the inactivation process is complete, the pH is adjusted to 7.0-7.5 with acid or base as mentioned above. The inactivated virus fluids are stored at 2-8° C. until further processing.

In embodiments, the immunogenic compositions described herein further include a pharmaceutically acceptable carrier and/or an adjuvant. The pharmaceutically acceptable carriers useful in this invention are conventional. Remington's Pharmaceutical Sciences (18th ed.; Mack Publishing Company, Eaton, Pa., 1990), describes compositions and formulations suitable for pharmaceutical delivery of one or more antigenic components, such as one or more HA- or HA fragment-encoding nucleic acid molecules, HA-expressing vectors, and/or HA proteins combined with various pharmaceutically acceptable additives, as well as a dispersion base or vehicle. Desired additives include, but are not limited to, pH control agents, such as arginine, sodium hydroxide, glycine, hydrochloric acid, citric acid, and the like. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), reducing agents (e.g., glutathione), and preservatives (e.g., antimicrobials, and antioxidants) can be included. In general, the nature of the carrier will depend on the particular mode of administration being employed.

Various adjuvants may be used to increase the immunological response to the immunogenic compositions described herein. By “adjuvant” is intended a compound or mixture that enhances the immune response to an antigen. An adjuvant can serve as a tissue depot that slowly releases the antigen, and also as a lymphoid system activator that non-specifically enhances the immune response. Often, a primary challenge with an antigen alone, in the absence of an adjuvant, will fail to elicit a humoral or cellular immune response. Adjuvants include, but are not limited to, complete Freund's adjuvant, incomplete Freund's adjuvant, lipopolysaccharide and lipopolysaccharide derivatives (e.g., MPL™, 3-O-deacylated monophosphoryl lipid A; Corixa, Hamilton Ind.), saponin and saponin derivatives (e.g., QS21), mineral gels such as aluminum hydroxide (e.g., AMPHOGEL™, Wyeth Laboratories, Madison, N.J.), surface active substances such as lysolecithin, pluronic polyols, polyanions, peptides, oil or hydrocarbon emulsions, keyhole limpet hemocyanins, dinitrophenol, CpG oligonucleotides, BCG (bacille Calmette-Guerin, the attenuated Mycobacterium bovis bacterium) and Corynebacterium parvum. Development of vaccine adjuvants for use in humans is reviewed in Singh et al. (Nat. Biotechnol. 17:1075-1081, 1999). Preferably, the adjuvant is pharmaceutically acceptable.

The immunogenic compositions described herein can be dispersed in a base or vehicle, which can include a hydrophilic compound having a capacity to disperse an immunogenic composition and any desired additives. The base can be selected from a wide range of suitable compounds, including but not limited to, copolymers of polycarboxylic acids or salts thereof, carboxylic anhydrides (e.g., maleic anhydride) with other monomers (e.g., methyl (meth)acrylate, acrylic acid and the like), hydrophilic vinyl polymers, such as polyvinyl acetate, polyvinyl alcohol, polyvinylpyrrolidone, cellulose derivatives, such as hydroxymethylcellulose, hydroxypropylcellulose and the like, and natural polymers, such as chitosan, collagen, sodium alginate, gelatin, hyaluronic acid, and nontoxic metal salts thereof. Often, a biodegradable polymer is selected as a base or vehicle, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters and the like can be employed as vehicles. Hydrophilic polymers and other vehicles can be used alone or in combination, and enhanced structural integrity can be imparted to the vehicle by partial crystallization, ionic bonding, cross-linking and the like. The vehicle can be provided in a variety of forms, including, fluid or viscous solutions, gels, pastes, powders, microspheres and films for direct application to a mucosal surface.

The immunogenic compositions can be combined with the base or vehicle according to a variety of methods, and release of the immunogenic compositions can be by diffusion, disintegration of the vehicle, or associated formation of water channels. In some circumstances, the immunogenic compositions are dispersed in microcapsules (microspheres) or nanocapsules (nanospheres) prepared from a suitable polymer, for example, isobutyl 2-cyanoacrylate (see, e.g., Michael et al., J. Pharmacy Pharmacol. 43:1-5, 1991), and dispersed in a biocompatible dispersing medium, which yields sustained delivery and biological activity over a protracted time. The immunogenic compositions of the invention can alternatively contain as pharmaceutically acceptable vehicles substances required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, and triethanolamine oleate.

In embodiments, the immunogenic compositions described herein can be administered in a time release formulation, for example in a composition which includes a slow release polymer. These compositions can be prepared with vehicles that will protect against rapid release, for example a controlled release vehicle such as a polymer, microencapsulated delivery system or bioadhesive gel. Prolonged delivery in various compositions of the invention can be brought about by including in the composition agents that delay absorption, for example, aluminum monostearate hydrogels and gelatin. When controlled release formulations are desired, controlled release binders suitable for use in accordance with the invention include any biocompatible controlled release material which is inert to the active agent and which is capable of incorporating the immunogenic compositions described herein. Numerous such materials are known in the art. Useful controlled-release binders are materials that are metabolized slowly under physiological conditions following their delivery (e.g., at a mucosal surface, or in the presence of bodily fluids). Appropriate binders include, but are not limited to, biocompatible polymers and copolymers well known in the art for use in sustained release formulations. Such biocompatible compounds are non-toxic and inert to surrounding tissues, and do not trigger significant adverse side effects, such as nasal irritation, immune response, inflammation, or the like. They are metabolized into metabolic products that are also biocompatible and easily eliminated from the body.

The influenza virus antigenic components contemplated herein include antigenic components (e.g., viral transmembrane and surface proteins and glycoproteins, such as HA and NA proteins and antigenic portions/fragments thereof) encoded by any influenza virus of interest. Fusion proteins are also contemplated that include a heterologous amino acid sequence chemically linked to an antigenic component. Exemplary heterologous sequences include short amino acid sequence tags (such as six histidine residues), as well a fusion of other proteins (such as c-myc or green fluorescent protein fusions). Epitopes of the antigenic components that are recognized by an antibody or that bind the major histocompatibility complex, and can be used to induce a specific immune response, are also contemplated. By “epitope” is intended an antigenic determinant. These are particular chemical groups or contiguous or non-contiguous peptide sequences on a molecule that are antigenic, that is, that elicit a specific immune response. An antibody binds a particular antigenic epitope based on the three dimensional structure of the antibody and the matching (or cognate) epitope.

As used herein, the term “antibodies” includes intact immunoglobulins as well as a number of well-characterized fragments. For instance, Fabs, Fvs, and single-chain Fvs (SCFvs) that bind to target protein (or epitope within a protein or fusion protein) would also be specific binding agents for that protein (or epitope). These antibody fragments are defined as follows: (1) Fab, the fragment which contains a monovalent antigen-binding fragment of an antibody molecule produced by digestion of whole antibody with the enzyme papain to yield an intact light chain and a portion of one heavy chain; (2) Fab′, the fragment of an antibody molecule obtained by treating whole antibody with pepsin, followed by reduction, to yield an intact light chain and a portion of the heavy chain; two Fab′ fragments are obtained per antibody molecule; (3) (Fab′)2, the fragment of the antibody obtained by treating whole antibody with the enzyme pepsin without subsequent reduction; (4) F(ab′)2, a dimer of two Fab′ fragments held together by two disulfide bonds; (5) Fv, a genetically engineered fragment containing the variable region of the light chain and the variable region of the heavy chain expressed as two chains; and (6) single chain antibody, a genetically engineered molecule containing the variable region of the light chain, the variable region of the heavy chain, linked by a suitable polypeptide linker as a genetically fused single chain molecule. Methods of making these fragments are routine (see, for example, Harlow and Lane, Using Antibodies: A Laboratory Manual, CSHL, New York, 1999). Antibodies for use in the methods of this invention can be monoclonal or polyclonal.

The term “antigenic portion” (e.g., of an antigenic component, such as an HA protein) includes any part, piece, or segment that is representative of, or derived from, a whole and retains the antigenic characteristics (e.g., the ability to elicit a specific immune response, such as a cell-mediated immune response, a humoral immune response, or both) of the whole. As used herein, “antigenic diversity” in reference to a target population of influenza virus strains refers to the variability of antigens (e.g., HA- or NA-derived) expressed by a population of influenza viruses. By “target population” is intended a set of influenza viruses relevant to a particular group of subjects for whom protective immunity is desired, and includes viruses of a particular subtype (e.g., H₃N₂, H₁N₁, H₅N₁, H₅N₂, H₉N₂, H₆N₁, or H₇N₇), type (e.g., influenza A or B viruses), geographic region (i.e., a mixture of types and subtypes), or all influenza viruses that infect a particular subject (e.g., humans).

Exemplary methods for determining the range of antigenic diversity among an antigenic component within a target population of influenza virus strains include, but are not limited to, determining the amino acid sequence for at least a portion of the antigenic regions of the antigenic component (e.g., by sequencing DNA or cDNA derived from viral RNA encoding the same), antigenic analysis of the antigenic component (e.g., hemagglutination-inhibition, microneutralization, or enzyme-linked immunosorbent assay), and in vivo testing in an animal or human subject.

Sequencing the antigenic regions of antigenic components (e.g., HA proteins) from a target population of influenza viruses enables the construction of phylogenetic trees, which facilitate a visual display of the relationships between viruses or portions of viruses. Viruses which have a similar sequence will be on the same “branch” of the tree; the closer two viruses are to one another, the more related they are. Viruses tend to cluster together in the tree in groupings called “clades.” Often these clades represent antigenically distinct groups of viruses. Viruses within a clade are similar, while viruses in different clades are less similar. Methods of protein sequencing are well known in the art (e.g., Edman degradation). As is well known in the art, the sequence of a protein (e.g., an HA or NA protein) can also be deduced from the mRNA or gene encoding the protein.

Antigenic analysis of antigenic components (e.g., HA proteins) can be performed by a number of methodologies known to one of ordinary skill in the art, such as, for example, microneutralization (see, e.g., Casals, J. Immunological techniques for animal viruses in Methods in Virology. Volume III. Maramorosch, K. and Koprowski, H. (eds.), Academic Press, NY, 113-94, 1967; Zielinska et al., Virology J. 2:84, 2005), enzyme-linked immunosorbent assay (ELISA; Engvall, Meth. Enzymol., 70:419-39, 1980) and derivatives thereof, and hemagglutination-inhibition.

Neutralization assays can be used to detect the presence of antibodies directed against a specific antigenic component in a preparation (e.g., serum) or to characterize an antigenic component using antibodies to probe its antigenicity. Viruses such as influenza will undergo replication in certain cell lines (e.g., Madin-Darby canine kidney (MDCK) cells) producing viral proteins, viral particles and cytopathic effects on the cell monolayer. In a neutralization assay, a preparation that may contain virus expressing the antigenic component that is being examined is mixed with a preparation that may contain antibody that may recognize that antigenic component. If antibodies present in the mixture can bind in sufficient number and to sufficiently important antigenic regions as to prevent replication of the virus in the cells, then production of viral proteins, whole viruses and generation of cytopathic effects do not occur. The amount of virus or antibody in the initial preparations can be estimated by titration, and the specificity of the antibody or the antigenic component can be determined by incubation with multiple partners.

Detection of neutralization can be done by numerous methods to detect the virus, viral proteins or cytopathic effect. In the case of the standard microneutralization assay, the assay is performed in 96 well plates suitable for use in a microplate reader such as that used for ELISA. After mixture of the antibody containing and antigen containing preparations, and incubation on the cells for a period of time sufficient to induce the desired effect (e.g., 18-22 hours), the cells are washed, and antibody specific for the nucleoprotein of influenza viruses is added as a primary antibody. A secondary antibody conjugated to a colorimetric, fluorescent or luminescent substrate is added, and light or change in color is detected using an ELISA plate reader.

Enzyme immunoassays such as ELISA can be readily adapted to accomplish antigenic analysis of antigenic components (e.g., HA proteins) according to the methods of this invention. As understood by one of skill in the art, enzyme immunoassays can be used either for antigenic analysis of the HA by incubating unknown viruses with known antibodies, or for characterization of antibodies from unknown sera using known viruses. In the methodology of determining what viruses are in a population, the former would be appropriate; for evaluating the immune response, the latter would be appropriate.

An ELISA method effective for the detection of soluble antigenic components is the direct competitive ELISA. This method is most useful when a specific antigenic component antibody (e.g., an HA-specific antibody) and purified antigenic component (e.g., an HA antigen) are available. Briefly: 1) coat a substrate (e.g., a microtiter plate) with a sample suspected of containing an antigenic component; 2) contact the bound antigenic component with an antigenic component-specific antibody bound to a detectable moiety (e.g., horseradish peroxidase enzyme or alkaline phosphatase enzyme); 3) add purified inhibitor antigenic component; 4) contact the above with the substrate for the enzyme; and 5) observe/measure inhibition of color change or fluorescence and quantitate antigenic component concentration (e.g., using a microtiter plate reader).

An additional ELISA method effective for the detection of soluble antigenic components is the antibody-sandwich ELISA. This method is frequently more sensitive in detecting antigen than the direct competitive ELISA method. Briefly: 1) coat a substrate (e.g., a microtiter plate) with an antigenic component-specific antibody; 2) contact the bound antigenic component antibody with a sample suspected of containing an antigenic component; 3) contact the above with antigenic component-specific antibody bound to a detectable moiety (e.g., horseradish peroxidase enzyme or alkaline phosphatase enzyme); 4) contact the above with the substrate for the enzyme; and 5) observe/measure color change or fluorescence and quantitate antigenic component concentration (e.g., using a microtiter plate reader).

An ELISA method effective for the detection of cell-surface antigenic components is the direct cellular ELISA. Briefly, cells suspected of exhibiting a cell-surface antigenic component are fixed (e.g., using glutaraldehyde) and incubated with an antigenic component-specific antibody bound to a detectable moiety (e.g., horseradish peroxidase enzyme or alkaline phosphatase enzyme). Following a wash to remove unbound antibody, substrate for the enzyme is added and color change or fluorescence is observed/measured.

Methods for inducing an immune response in a subject against an influenza virus are also encompassed by the present invention, and include administering to a subject a therapeutically effective amount of the immunogenic compositions described herein (e.g., an antigenic component-expressing vector, such as an HA-expressing vector), typically combined together with one or more pharmaceutically acceptable vehicles and, optionally, other therapeutic ingredients (e.g., antibiotics, or anti-inflammatories). In some embodiments, a therapeutically effective amount of one or more viral proteins or antigenic portions thereof (e.g., HA and/or NA proteins), or one or more live attenuated viruses, derived from the target population of influenza virus strains is further administered to the subject. Such a prime:boost strategy (or coordinate vaccination protocol) is used to enhance an immune response elicited by the antigenic component-expressing vector alone (e.g., an HA-expressing vector). Typically, when a prime:boost strategy is employed, the antigenic component-expressing vector immunogen and the viral protein/live attenuated virus booster are administered coordinately, in a specified temporal sequence (e.g., separated by two weeks, three weeks, one month, three months, etc.).

Within the methods of the invention, the immunogenic compositions can be administered to subjects by a variety of mucosal administration modes, including by oral, rectal, upper respiratory tract (i.e., the nasal cavity, pharynx, larynx, and trachea), intrapulmonary, or transdermal delivery. Mucosal administration can be by way of spray, droplet, aerosol, or by topical delivery. Optionally, the immunogenic compositions can be administered by non-mucosal routes, including by intramuscular, subcutaneous, subdermal, intravenous, intra-atrial, intra-articular, intraperitoneal, or parenteral routes.

The immunogenic compositions disclosed herein can be administered to the subject in a single bolus delivery, via continuous delivery (e.g., continuous transdermal, mucosal or intravenous delivery) over an extended time period, or in a repeated administration protocol (e.g., by a weekly or monthly, repeated administration protocol). The therapeutically effective dosage of the immunogenic composition can be provided as repeated doses within a prolonged prophylaxis or treatment regimen, that will yield clinically significant results to alleviate one or more symptoms or detectable conditions associated with a targeted disease or condition as set forth herein. Determination of effective dosages in this context is typically based on animal model studies followed up by human clinical trials and is guided by administration protocols that significantly reduce the occurrence or severity of targeted disease symptoms or conditions in the subject. Suitable models in this regard include, for example, murine, rat, ferret, porcine, feline, non-human primate, and other accepted animal model subjects known in the art.

Alternatively, effective dosages can be determined using in vitro models (e.g., immunologic and histopathologic assays). Using such models, only ordinary calculations and adjustments are required to determine an appropriate concentration and dose to administer a therapeutically effective amount of the immunogenic composition (e.g., amounts that are effective to elicit a desired immune response or alleviate one or more symptoms of a targeted disease). In alternative embodiments, an effective amount or effective dose of the immunogenic composition may simply inhibit or enhance one or more selected biological activities correlated with a disease or condition, as set forth herein, for either therapeutic or diagnostic purposes.

The actual dosage of the immunogenic composition will vary according to factors such as the disease indication and particular status of the subject (e.g., the subject's age, size, fitness, extent of symptoms, susceptibility factors, and the like), time and route of administration, other drugs or treatments being administered concurrently, as well as the specific pharmacology of the immunogenic composition for eliciting the desired activity or biological response in the subject. Dosage regimens can be adjusted to provide an optimum prophylactic or therapeutic response. When provided prophylactically, the immunogenic compositions of the invention are provided in advance of any symptom. Prophylactic administration serves to prevent or ameliorate any subsequent infection with an influenza virus. The immunogenic compositions of the invention can thus be provided prior to the anticipated exposure to one or more influenza virus strains, so as to attenuate the anticipated severity, duration or extent of an infection and/or associated disease symptoms, after exposure or suspected exposure to the viruses, or after the actual initiation of an infection.

Upon administration of an immunogenic composition of the invention (e.g., via injection, aerosol, oral, topical or other route), the immune system of the subject typically responds to the immunogenic composition by producing antibodies specific for multiple influenza viruses within the target population. Such a response signifies that an immunologically effective dose of the immunogenic composition was delivered. An immunologically effective dosage can be achieved by single or multiple administrations. For each particular subject, specific dosage regimens can be evaluated and adjusted over time according to the individual need and professional judgment of the person administering or supervising the administration of the immunogenic composition. In some embodiments, the antibody response of a subject administered the immunogenic compositions of the disclosure will be determined in the context of evaluating effective dosages/immunization protocols. In most instances it will be sufficient to assess the antibody titer in serum or plasma obtained from the subject. Decisions as to whether to administer booster inoculations and/or to change the amount of the immunogenic composition administered to the subject can be at least partially based on the antibody titer level. The antibody titer level can be based on, for example, an immunobinding assay which measures the concentration of antibodies in the serum which bind to a specific antigen, for example, an HA or NA antigen. The ability to neutralize in vitro and in vivo biological effects of target influenza virus strains can also be assessed to determine the effectiveness of the treatment.

Typical subjects intended for treatment with the immunogenic compositions and methods of the present invention include humans, as well as non-human primates and other animals. To identify subjects for prophylaxis or treatment according to the methods of the invention, accepted screening methods are employed to determine risk factors associated with a targeted or suspected disease or condition (e.g., influenza), or to determine the status of an existing disease or condition in a subject. These screening methods include, for example, conventional work-ups to determine environmental, occupational and other such risk factors that may be associated with the targeted or suspected disease or condition, as well as diagnostic methods, such as various ELISA and other immunoassay methods, which are available and well known in the art to detect and/or characterize disease-associated markers. These and other routine methods allow the clinician to select patients in need of therapy using the methods and immunogenic compositions of the invention. In accordance with these methods and principles, an immunogenic composition can be administered according to the teachings herein as an independent prophylaxis or treatment program, or as a follow-up, adjunct or coordinate treatment regimen to other treatments, including conventional influenza vaccines.

The instant invention also includes kits, packages and multi-container units containing the herein described immunogenic compositions, active ingredients, and/or means for administering the same for use in the prevention and treatment of influenza in subjects. In one embodiment, these kits include a container or formulation that contains one or more of the immunogenic compositions and/or other active agents described herein. In one example, this component is formulated in a pharmaceutical preparation for delivery to a subject. The immunogenic compositions are optionally contained in a bulk dispensing container or unit or multi-unit dosage form. Optional dispensing means can be provided, for example a pulmonary or intranasal spray applicator. Packaging materials optionally include a label or instruction indicating for what treatment purposes and/or in what manner the immunogenic composition packaged therewith can be used.

The subject matter of the present disclosure is further illustrated by the following non-limiting examples.

### EXAMPLES

### Example 1. Hemagglutination Assay

Subunit protein (sHA) was generated and isolated as described above. Virus (control) and sHA (test) were tested in duplicate using serial 2-fold dilutions in a microtiter plate, with a final volume of 50 μl. An equal volume of turkey red blood cell suspension was added, and the contents was mixed and allowed to incubate for 1-2 hours at room temperature (25° C.). The plates were read for hemagglutination, where the titer was recorded as the inverse of the dilution in which the last dilution where both wells exhibiting hemagglutination was noted. The titer was recorded in HAU (hemagglutination units) per 50 μl (see Table 2).

TABLE 2
Hemagglutination Assay
Unclarified Antigen

Titer (HAU/50 μl)

| GOI | Pre BPL | Post BPL |
|---|---|---|
| H3 | ≥1024^{€} | 64+ |
| H3 | 5124^{€} | 80+ |
| H3 | ≥1024^{€} | 16+ |
| H3 | 1280^{€} | 40^{#} |
| H1 calde 3a | 80^{#} | 40^{#} |
| H3 | 5120^{€} | 5120^{€} |
| H3 | 5120^{€} | 5120^{€} |
| H3 | ≥10240^{€} | 80^{#} |
| H1 | 2560^{€} | 1280^{€} |
| H1 npdm | 40^{#} | 40^{#} |
| H1 alpha | 640^{€} | 80^{#} |
| H3 | ≥10240^{€} | 1280^{€} |
| H3 | ≥10240^{€} | 2560^{€} |
| H3 | ≥10240^{€} | 80^{#} |
| H1 clade 3a | 1280+^{€} | 640+^{€} |
| H3N2 | ≥10240^{€} | ≥10240^{€} |
| H1 | ≥10240^{€} | ≥10240^{€} |
| H3 | ≥10240^{€} | 5120+^{€} |
| H1 npdm | 320+^{€} | 320+^{€} |
| H3 alpha | 2560^{€} | 2560^{€} |
| H3 | 640^{€} | 220+^{€} |
| H3 | ≥10240^{€} | 2560^{€} |
| H3 | 5120^{€} | 1260+^{€} |
| H3 | 2560^{€} | 2560^{€} |
| H3 | 2560^{€} | 2560^{€} |
| H3 | 2560^{€} | 2560^{€} |
| H3 | 2560^{€} | 1280+^{€} |
| H3 | 2560^{€} | 2560^{€} |
| ORF2 capsid | 40^{#} | <10^{#} |
| H1 (OH) | 2560+^{€} | 1280+^{€} |
| H1 (NC) | 640^{€} | 160+^{#} |
| H1 | 1280^{€} | 320^{€} |
| H5 | ≥10240^{€} | ≥10240^{€} |
| H1 clade 3a | 160+^{#} | 40+^{#} |
| H1 clade 3a | 160+^{#} | 80^{#} |
| H3 | ≥5120^{€} | 5120^{€} |
| H1 (NC) | 80+^{#} | 40^{#} |
| H1 (NC) | 10+^{#} | 10^{#} |
| H1 | 640^{€} | 320^{€} |
| H3N2 | 5120^{€} | 2560^{€} |
| H3N2 | 2560^{€} | 1280^{€} |
| H1 (OH) | 80+^{#} | 80+^{#} |
| H3 | 640+^{€} | 320+^{€} |
| H1 npdm | 20^{#} | 20^{#} |
| H1 npdm | <10+^{#} | <10+^{#} |
| H1 alpha | 40+^{#} | 20+^{#} |
| H1 clade 3a | 40+^{#} | 40+^{#} |
| H1 clade 3a | 40+^{#} | 20+^{#} |
| H1 (NC) | 320^{€} | 160^{#} |
| H1 (NC) | 320^{€} | 160^{#} |
| H3 | 640^{€} | 320^{€} |
| H3 | 640^{€} | 320^{€} |
| H1 npdm | 40+^{#} | 40+^{#} |
| H1 npdm | 40+^{#} | 40+^{#} |
| H1 alpha | 640^{€} | 320+^{€} |
| H1 alpha | 640^{€} | 160^{#} |
| H1 (NC) | 320^{€} | 160^{#} |
| H1 (NC) | 2560^{€} | 640^{€} |
| H1 (NC) | 2560^{€} | 2560^{€} |
| H1 | 320^{€} | 160^{#} |
| H1 npdm | 80^{#} | 80^{#} |
| H1 alpha | 1280^{€} | 640^{€} |
| H1 alpha | 320+^{€} | 320+^{€} |
| H1 clade 3a | 320+^{€} | 160+^{#} |
| H1 clade 3a | 320+^{€} | 160+^{#} |
| H1 | 5120^{€} | 2560+^{€} |
| H1 | 5120^{€} | 2560+^{€} |
| H3 | ≥10240^{€} | 5120^{€} |
| H3 | 5120^{€} | 5120^{€} |
| H1 alpha | 80^{#} | 10^{#} |
| H1 | ≥10240^{€} | ≥5120^{€} |
| H1 | 320^{€} | 80^{#} |
| H1 clade 3a | 640+^{€} | 160+^{#} |
| H3 | ≥10240^{€} | ≥5120^{€} |
| H1 | ≥10240^{€} | ≥10240^{€} |
| H1 | ≥10240^{€} | ≥10240^{€} |
| H1 npdm | 540^{€} | 320^{€} |
| H1 npdm | 320+^{€} | 320^{€} |
| H3 | ≥10240^{€} | 5120^{€} |
| H3 | ≥10240^{€} | 5120^{€} |
| H5 | 320+^{€} | 2560+^{€} |

^{€}Positive Titer
^{#}Negative Titer
Positive Titer Cutoff = 320 HAU/50 μl.
BPL = Beta-propiolactone.

As may be seen in the Table, BPL addition (viral fluid inactivation) can affect titer.

### Example 2. Hemagglutination Inhibition Assay

sHA was generated and isolated as described in Example 1. Each sera sample was treated with a receptor destroying enzyme (RDE, e.g., from Hardy Diagnostics, Santa Maria, CA) or 10% Kaolin suspension, followed by treatment with turkey red blood cells for non-specific agglutinins. Virus (control) and sHA (test) were tested in duplicate using serial 2-fold dilutions in a microtiter plate, the final volume of 50 μl. Indicator virus/antigen was added to all wells at a concentration of 4 HAU per 25 μl. Plates contents were mixed and allowed to incubate for 30 minutes. After incubation, 50 μl of a 1% turkey red blood cell suspension was added. The contents of the plates were mixed and allowed to incubate for 1-2 hours at room temperature. The plates were read for hemagglutination, where the titer was recorded as the inverse of the dilution in which the last dilution where both wells exhibiting hemagglutination was noted. The titer was recorded in HAU (hemagglutination units) per 25 μl. (See Table 3).

TABLE 3
IAV H5 Antigen on various species of Sera
HAI Results - Titers (HAU/25/ul)
RDE Treatment 294/58

| Key | Titer | Species |
|---|---|---|
| 1 | <10 | Bovine |
| 2 | <10 | Bovine |
| 3 | <10 | Bovine |
| 4 | <10 | Bovine |
| 5 | <10 | Bovine |
| 6 | <10 | Bovine |
| 7 | <10+ | Bovine |
| 8 | <10 | Bovine |
| 9 | <10+ | Bovine |
| 10 | <10 | Bovine |
| 11 | <10 | Bovine |
| 12 | <10 | Bovine |
| 13 | <10 | Bovine |
| 14 | <10 | Bovine |
| 15 | <10 | Bovine |
| 16 | <10 | Swine |
| 17 | <10 | Swine |
| 18 | <10 | Swine |
| 19 | <10 | Swine |
| 20 | <10 | Swine |
| 21 | <10 | Swine |
| 22 | <10 | Swine |
| 23 | <10 | Swine |
| 24 | <10 | Swine |
| 25 | <10 | Swine |
| 26 | <10 | Bovine |
| 27 | <10 | Bovine |
| 28 | <10 | Bovine |
| 29 | <10 | Bovine |
| 30 | <10 | Bovine |
| 31 | <10 | Sheep |
| 32 | <10 | Sheep |
| 33 | <10 | Sheep |
| 34 | <10 | Sheep |
| 35 | <10 | Sheep |
| 36 | <10 | Deer |
| 37 | <10 | Deer |
| 38 | <10 | Deer |
| 39 | <10 | Deer |
| 40 | <10 | Deer |
| 41 | <10 | Bovine |
| 42 | <10 | Bovine |
| 43 | <10 | Bovine |
| 44 | <10 | Bovine |
| 45 | <10 | Bovine |
| 46 | <10 | Horse |
| 47 | <10 | FBS |
| 48 | <10 | Rabbit |

RDE Treatment 294/56

| Key | Treatment |
|---|---|
| 1 | 160 |
| 2 | 160 |
| 3 | 160 |
| 4 | 20 |
| 5 | 40 |
| 6 | 160 |
| 7 | 160 |
| 8 | 10+ |
| 9 | 160+ |
| 10 | 160 |
| 11 | 160 |
| 12 | 80++ |
| 13 | <10 |
| 14 | 160 |
| 15 | <10 |
| 7** | 640 |
| 8** | 80 |
| 29** | 320 |
| 48** | <10 |
| 49** | <10 |
| *PC-ID:20 | 320 |
| *NC-ID:52 | <10 |

*Bovine sample from Circle H (IA)
**Sample not RDE treated

### Example 2. HAI Method Using Potassium Periodate Pretreatment of Serum

Briefly, each serum sample was treated with potassium periodate, followed by heat inactivation at 56° C. for 30 minutes and nonspecific serum agglutinin removal by incubation with porcine red blood cells. Hemagglutination inhibition was tested in U-bottom plates. Each treated sera sample was tested in duplicate, in serial 2-fold dilutions starting at a 1:8 dilution, resulting in a final volume of 25 μl. Inactivated IAV-H5 antigen at a concentration of 4 HAU per 25 μl was added and incubated for 30 minutes at 37° C. Following incubation, 50 μl of 0.5% porcine red blood cells were added and incubated for 1.5-2 hours at 37° C. Plates were read using a magnified plate reader. Inhibition of hemagglutination by the test sera was defined as positive when erythrocytes settled into a well-defined ring or button on the bottom of the well. Serum HAI titers were expressed as the reciprocal of the highest dilution having complete inhibition of hemagglutination. Titer was recorded in HAU per 25 μl. (See Table 3).

TABLE 3
IAV H5 Ag on Bovine Sera
HAI Results - Titers (HAU/25 ul)

| Key | New | OD | Inter. |
|---|---|---|---|
| 1 | 320^{€} | 0.15 | Pos^{#} |
| 2 | 160^{€} | 0.17 | Pos^{#} |
| 3 | 160+^{€} | 0.19 | Pos^{#} |
| 4 | 320^{€} | 0.20 | Pos^{#} |
| 5 | 320+^{€} | 0.21 | Pos^{#} |
| 6 | 160^{€} | 0.21 | Pos^{#} |
| 7 | 320^{€} | 0.22 | Pos^{#} |
| 8 | 640^{€} | 0.22 | Pos^{#} |
| 9 | 160^{€} | 0.22 | Pos^{#} |
| 10 | 160^{€} | 0.22 | Pos^{#} |
| 11 | 160^{€} | 0.24 | Pos^{#} |
| 12 | 320+^{€} | 0.25 | Pos^{#} |
| 13 | 160+^{€} | 0.25 | Pos^{#} |
| 14 | 640^{€} | 0.25 | Pos^{#} |
| 15 | 160^{€} | 0.26 | Pos^{#} |
| 16 | 320^{€} | 0.27 | Pos^{#} |
| 17 | 640^{€} | 0.28 | Pos^{#} |
| 18 | 320^{€} | 0.29 | Pos^{#} |
| 19 | 80^{€} | 0.31 | Pos^{#} |
| 20 | 40 | 0.31 | Pos^{#} |
| 21 | 20 | 0.31 | Pos^{#} |
| 22 | 20 | 0.32 | Pos^{#} |
| 23 | 320^{€} | 0.35 | Pos^{#} |
| 24 | 160^{€} | 0.37 | Pos^{#} |
| 25 | 80^{€} | 0.41 | Pos^{#} |
| 26 | 20 | 0.42 | Pos^{#} |
| 27 | 40 | 0.43 | Pos^{#} |
| 28 | 10+ | 0.43 | Pos^{#} |
| 29 | 20 | 0.45 | Pos^{#} |
| 30 | 20+ | 0.46 | Pos^{#} |
| 31 | 20 | 0.47 | Pos^{#} |
| 32 | 10 | 0.47 | Pos^{#} |
| 33 | 10 | 0.49 | Pos^{#} |
| 34 | <10 | 0.51 | Neg |
| 35 | 20 | 0.53 | Neg |
| 36 | 20 | 0.55 | Neg |
| 37 | 10+ | 0.55 | Neg |
| 38 | 10+ | 0.56 | Neg |
| 39 | 20+ | 0.57 | Neg |
| 40 | 20 | 0.60 | Neg |
| 41 | <10 | 0.61 | Neg |
| 42 | 20+ | 0.65 | Neg |
| 43 | <10 | 0.69 | Neg |
| 44 | <10 | 0.71 | Neg |
| 45 | 20 | 0.72 | Neg |
| 46 | 20 | 0.75 | Neg |
| 47 | <10 | 0.76 | Neg |
| 48 | 20 | 0.76 | Neg |
| 49 | <10 | 0.77 | Neg |
| 50 | 10+ | 0.80 | Neg |
| 51 | 40 | 0.82 | Neg |
| 52 | 10 | 0.82 | Neg |
| 53 | <10 | 0.82 | Neg |
| 54 | <10 | 1.10 | Neg |
| 55 | <10 | 1.17 | Neg |
| 56 | 10 | 1.19 | Neg |
| 57 | <10 | 1.25 | Neg |
| 58 | <10 | 1.75 | Neg |

Although the invention has been described with reference to the above examples, it will be understood that modifications and variations are encompassed within the spirit and scope of the invention. Accordingly, the invention is limited only by the following claims.

I claim:

1. A hemagglutination inhibition assay (HIA) for identifying neutralizing antibody against avian influenza virus (IAV) comprising:
a) mixing sera containing antibody, a subunit protein derived from an avian influenza viral antigen, and red blood cells (RBCs) in the absence of live or attenuated avian virus;
b) allowing the mixture to stand for a sufficient time to allow for interaction between the three components, and
c) determining whether the RBCs aggregate.

2. The assay of claim 1, wherein where if aggregation of the RBCs is inhibited, then the subunit protein will produce neutralizing antibodies when used in a vaccine composition.

3. The assay of claim 2, wherein the subunit protein is an avian influenza surface antigen.

4. The assay of claim 3, wherein the subunit protein is hemagglutinin and/or neuraminidase.

5. The assay of claim 1, wherein the IAV strain is a highly pathogenic avian influenza (HPAI) type A or type B strain.

6. The assay of claim 5, wherein the IAV is selected from the group consisting of type B influenza, subtype H₃N₂, type A influenza, subtype H₁N₁, type A influenza, subtype H₅N₁, type A influenza, subtype H₅N₂, type A influenza, subtype H₉N₂, type A influenza, subtype H₆N₁, type A influenza, subtype H₇N₇.

7. The assay of claim 1, wherein the subunit protein is obtained from A/Common tern/ME/22-020831-002 original/2022 (H₅N₁) (GenBank No.: PQ709561.1).

8. The assay of claim 7, wherein the subunit protein is screened against antibody generated from IAV strains selected from the group consisting of A/swine/Missouri/A01410818/2013 (H₃N₁) (GenBank No.: KJ941368.1), A/swine/Nebraska/A02245325/2019 (H₃N₂) (GenBank No.: MN932087.1), A/swine/Iowa/A01432233/2012 (H₁N₁) (GenBank No.: KC436080.1), A/swine/Iowa/A02478455/2019 (H₃N₂) (GenBank No.: MK967587.1), A/swine/Missouri/A01410818/2013 (H₃N₁) (GenBank No.: KJ941268.1), A/swine/Ohio/A02431145/2019 (H₁N₂) (GenBank No.: MK714853.1), A/swine/North Carolina/A02431520/2019 (H₁N₂) (GenBank No.: MK799887.1), A/swine/North Carolina/A02479173/2020 (H₁N₁) (GenBank No.: MT367647.1), A/West Virginia/04/2020 (H₁) (GenBank No.: MT330463.1), A/swine/Iowa/A02479163/2020 (H₁N₂) (GenBank No.: MT476698.1), and A/swine/Iowa/A01566613/2014 (H₃N₂) (GenBank No.: KP247601.1).

9. The method of claim 1, wherein the antibody is collected from the sera of animals selected from the group consisting of swine, bovine, sheep, rabbit, deer, and equine subjects, and wherein said animal is infected by an IAV or naïve to such infection.

10. The method of claim 2, wherein a neutralizing antibody is positively identified if it has an HA assay titer of at least 320 HAU/50 μl.

11. A hemagglutination inhibition assay (HIA) for identifying neutralizing antibodies against avian influenza virus (IAV) comprising:
a) mixing sera containing antibody, a subunit protein derived from an avian influenza viral antigen, and red blood cells (RBCs) in a suitable container in the absence of live or attenuated avian virus, wherein the subunit protein is generated by recombinant means;
b) allowing the mixture to stand for a sufficient time to allow for interaction between the three components, and
c) determining whether the RBCs aggregate.

12. The assay of claim 11, wherein the recombinant means is a recombinant baculovirus protein expression system by infecting Spodoptera frugiperda insect cells (Sf9) with baculovirus containing heterologous nucleic acids encoding said subunit protein, and wherein a final sequence is optimized for expression in Sf9.

13. The assay of claim 12, wherein resulting viral fluids generated from infected Sf9 cells are inactivated with Beta-propiolactone (BPL).

14. The assay of claim 11, wherein the sera is combined with receptor destroying enzyme (RDE) or kaolin.

15. The assay of claim 11, wherein the RBCs are from turkey.

16. The assay of claim 11, wherein the IAV strain is a highly pathogenic avian influenza (HPAI) type A or type B strain.

17. The assay of claim 16, wherein the IAV is selected from the group consisting of type B influenza, subtype H₃N₂, type A influenza, subtype H₁N₁, type A influenza, subtype H₅N₁, type A influenza, subtype H₅N₂, type A influenza, subtype H₉N₂, type A influenza, subtype H₆N₁ type A influenza, subtype H₇N₇.

18. The assay of claim 11, wherein the subunit protein is obtained from A/Common tern/ME/22-020831-002 original/2022 (H₅N₁) (GenBank No.: PQ709561.1).

19. The assay of claim 18, wherein the subunit protein is screened against antibody generated from IAV strains selected from the group consisting of A/swine/Missouri/A01410818/2013 (H₃N₁) (GenBank No.: KJ941368.1), A/swine/Nebraska/A02245325/2019 (H₃N₂) (GenBank No.: MN932087.1), A/swine/Iowa/A01432233/2012 (H₁N₁) (GenBank No.: KC436080.1), A/swine/Iowa/A02478455/2019 (H₃N₂) (GenBank No.: MK967587.1), A/swine/Missouri/A01410818/2013 (H₃N₁) (GenBank No.: KJ941268.1), A/swine/Ohio/A02431145/2019 (H₁N₂) (GenBank No.: MK714853.1), A/swine/North Carolina/A02431520/2019 (H₁N₂) (GenBank No.: MK799887.1), A/swine/North Carolina/A02479173/2020 (H₁N₁) (GenBank No.: MT367647.1), A/West Virginia/04/2020 (H₁) (GenBank No.: MT330463.1), A/swine/Iowa/A02479163/2020 (H₁N₂) (GenBank No.: MT476698.1), and A/swine/Iowa/A01566613/2014 (H₃N₂) (GenBank No.: KP247601.1), and wherein the antibody is collected from the sera of animals selected from the group consisting of swine, bovine, sheep, rabbit, deer, and equine subjects, and wherein said animal is infected by an IAV or naïve to such infection.

20. The method of claim 11, wherein a neutralizing antibody is positively identified if it has an HA assay titer of at least 320 HAU/50 μl.
